(19) United States
(12) Patent Application Publication
(10) Pub. No.: US 2006/0206051 A1
(43) Pub. Date: Sep. 14, 2006

(54) Gas irrigator for surgical procedures

(76) Inventor: Dwight Antony Hamilton, Signal Mountain, TN (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/363,874

(22) Filed: Feb. 28, 2006

Related U.S. Application Data
(63) Continuation-in-part of application No. 11/251,609, filed on Oct. 14, 2005, now abandoned.
Application No. 11/251,609 is a continuation-in-part of application No. 11/069,280, filed on Mar. 1, 2005, now abandoned.

(51) Int. Cl. A61M 37/00

(52) U.S. Cl. 604/24; 606/228

(57) ABSTRACT

A gas application device for supplying a gas such as carbon dioxide to a surgical site greatly reduces the risk of embolism during surgery. In certain embodiments, the gas application device includes a gas conduit loop and gas outlets spaced evenly along the gas conduit loop. The gas conduit loop is supplied carbon dioxide gas through a gas inlet port. The supply of carbon dioxide gas can be filtered and humidified upstream of the gas conduit loop. The gas outlets are configured such that they each operate at substantially equal volume flow rates, promoting a uniform circumferential gas flow from the gas conduit loop. The gas outlets can include diffusers to create microcurrents in the gas flow exiting the gas outlets, thus promoting substantially uniform concentrations of carbon dioxide throughout the surgical site. The gas application device can additionally feature a movable gas applicator to locally direct a gas flow below wound level.

### CROSS-REFERENCE TO RELATED APPLICATIONS

This application is a continuation-in-part of U.S. patent application Ser. No. 11/251,609, filed Oct. 14, 2005, currently pending, entitled “GAS IRRIGATOR FOR SURGICAL PROCEDURES”, which is a continuation-in-part of U.S. patent application Ser. No. 11/069,280, filed Mar. 1, 2005, currently pending, entitled “CARBON DIOXIDE APPLICATOR FOR USE WITH SURGICAL PROCEDURES.”

This application hereby incorporates the above-identified applications by reference in their entirety.

### BACKGROUND OF THE INVENTION

1. Field of the Invention

The present inventions relate to gas application devices generally, and more specifically to gas application devices to locally displace air at a surgical site.

2. Description of the Related Art

Air has long been recognized as the bane of heart surgery. Air has only limited solubility in blood. Unfortunately, air bubbles are often introduced into the circulatory system during heart surgeries. Upon reintroduction of blood, these air bubbles are carried by blood flow until they reach blood vessels that are too small for the bubbles to continue to flow. The bubbles then lodge and obstruct local blood circulation until they can be dissolved into the blood. This event, known as an embolism or air embolism, can have deadly consequences: if an embolism occurs in a blood vessel in the heart, it often results in a heart attack; and if an embolism occurs in a blood vessel in the brain, it often results in a localized stroke.

Various de-airing techniques for use after completing a surgical procedure have been employed to reduce the risk of embolism. These techniques include mechanical manipulation and venting of the heart (or other surgical site) to remove trapped air and aspiration of an air bubble by inserting a needle through tissue containing a lodged air bubble.

It has been found that carbon dioxide is significantly more soluble in blood than air. Carbon dioxide is believed to be at least ten times more soluble in blood than air. Additionally, it has been found that application of carbon dioxide to a surgical site can inhibit bacterial growth, thus reducing the risk of infection. Various techniques have been employed by heart surgeons to exploit the relatively high solubility of carbon dioxide in an attempt to reduce air embolism during surgery. Many heart surgeons began using a source of carbon dioxide pumped through a tube to an end of the tube secured to the patient or to a structure such as a retractor. Often, the end of the tube was placed below wound level to apply carbon dioxide as closely as possible to the surgical site. This placement below the wound level created an obstacle for a medical professional such as a surgeon working in the surgical site. When used in a heart surgery environment, the local concentrations of carbon dioxide throughout the pericardial cavity can vary widely as the single hose-like gas source produces currents and eddies of carbon dioxide within the surgical site.

Thus, the conventional single tube supplying carbon dioxide to the surgical site does not displace all of the air from the surgical site and does not provide relatively uniform carbon dioxide concentrations throughout the surgical site. Notwithstanding the uneven carbon dioxide concentrations created by a single-tube gas source, the introduction of carbon dioxide into the surgical site by a single tube markedly reduces the size and number of air bubbles introduced into the circulatory system as viewed on a transesophageal echocardiogram machine.

Attempts have been made to overcome the limitations of carbon dioxide delivery via a single tube. But these attempts have not adequately addressed the previously discussed limitations and have presented additional shortcomings. Certain of these attempts have included ring structures through which carbon dioxide is supplied from a single gas inlet and circumferentially arranged nozzles that expel the gas into the surgical site. However, in operation, these arrangements suffer a lack of uniformity in carbon dioxide concentration because nozzles closest to the gas inlet will expel most of the gas supply while nozzles far from the inlet will expel very little gas. Moreover, several of these attempts have positioned the nozzles below wound level where they present an obstacle to surgery. Other attempts that placed nozzles at wound level have likewise presented obstacles to surgery as they have not easily permitted usage of a retractor, sutures, or other medical implements commonly used in an open chest surgical procedure and many other surgical procedures. Also, previous wound-level devices have included nozzles configured to create a laminar flow, thus creating a carbon dioxide “cap” over a surgical site without displacing a significant volume of air from the surgical site. Therefore, there is a need for a gas application device for use during surgery that does not present an obstacle to a medical professional working in the surgical site. There is also a need for a gas application device that provides a substantially uniform circumferential flow of gas to displace air present in the surgical site.

### SUMMARY OF THE INVENTION

Various embodiments of the gas application devices described herein overcome the above-mentioned shortcomings of the prior art and provide further advantages more fully discussed herein. In various embodiments described herein, a gas application device can be used to create a substantially uniform gas flow circumferentially about a surgical site. The gas flow can be diffused into microcurrents to displace air and promote a uniform concentration of gas throughout the surgical site. The gas application device can be shaped and configured to extend around a surgical site while allowing a medical professional substantially unobstructed access to the site. Moreover, a movable gas applicator can be fluidly coupled to the gas application device to allow a medical professional to directly apply a flow of gas locally to the surgical site below wound level.

In certain embodiments a gas application device for use in surgery is provided. The gas application device can comprise: a gas conduit shaped and configured as a loop to overlie a pericardial cavity of a patient; a gas inlet port fluidly coupled to the gas conduit and configured to connect to a gas source; a plurality of gas outlets substantially evenly spaced along the gas conduit; a plurality of flow limiters, each of the plurality of flow limiters fluidly coupled to a corresponding one of the plurality of gas outlets; a plurality of diffusers, each of the plurality of diffusers fluidly coupled to a corresponding one of the plurality of gas outlets; a draping sheet mounted to an upper surface of the gas conduit and extending generally radially outward from the loop shaped gas conduit; and a plurality of suture holders formed in an upper surface of the gas conduit. The flow limiters are configured to maintain substantially even volumetric flow rates from each of the plurality of gas outlets. The diffusers are configured to create microcurrents in a flow of gas exiting the outlets.

In some embodiments, a gas application device comprising a support structure and a plurality of gas outlets arranged along the support structure is provided. Each of the gas outlets is configured to operate at approximately the same volume flow rate. In some embodiments, a gas application device for use in surgery comprising a gas conduit loop and a plurality of gas outlets disposed towards an interior of the loop is provided.

In some embodiments, a gas application device for use in surgery comprising a plurality of gas outlets disposed on a gas applicator structure, a plurality of feeder tubes, and a gas inlet port is provided. Each feeder tube corresponds to one of the plurality of gas outlets. Each of the plurality of feeder tubes is substantially the same length as each of the other of the plurality of feeder tubes. The gas inlet port is configured to accept an incoming stream of gas and distribute the incoming stream substantially evenly among the plurality of feeder tubes.

In some embodiments, a method for irrigating a surgical site with a gas is provided. The method comprises the steps of providing a gas application device, positioning the gas application device proximate the surgical site, and establishing a flow of gas to a gas conduit of the gas application device. The gas application device comprises a gas conduit and a plurality of gas outlets arranged along the gas conduit, wherein each of the gas outlets is configured to operate at approximately a same volume flow rate as each other of the plurality of gas outlets. The flow of gas to the gas conduit is established such that the flow of gas exits the gas outlets.

For purposes of summarizing the inventions and the advantages achieved over the prior art, certain objects and advantages of the inventions have been described above and further described below. Of course, it is to be understood that not necessarily all such objects or advantages can be achieved in accordance with any particular embodiment of the inventions.

### BRIEF DESCRIPTION OF THE DRAWINGS

FIG. 1 is a top view of one embodiment of a gas application device;

FIG. 2 is a top view of a gas application device as applied to an open surgical site in the thoracic cavity;

FIG. 2A is a cross-sectional view of the gas application device of FIG. 2 including certain embodiments of suture holder grooves;

FIG. 2B is a cross-sectional view of the gas application device of FIG. 2 including other embodiments of suture holder grooves;

FIG. 3 is a top view of another embodiment of a gas application device;

FIG. 4 is a cross-sectional view of the gas application device of FIG. 3 along the line 4-4;

FIG. 5 is a cross-sectional view of the gas application device of FIG. 3 along the line 5-5;

FIG. 6 is a cross-sectional view of the gas application device of FIG. 5 along the line 6-6;

FIG. 7 is a cross-sectional view of an embodiment of flow limiter and diffuser of a gas application device;

FIG. 8 is a cross-sectional view of another embodiment of flow limiter and diffuser of a gas application device;

FIG. 9 is an enlarged cross-sectional view of the flow limiter of FIG. 8 along line 9-9 in a closed position;

FIG. 10 is an enlarged cross-sectional view of the flow limiter of FIG. 8 in an open position;

FIG. 11 is a top view of a gas application device in an embodiment having a movable gas applicator;

FIG. 12 is a perspective view of the movable gas applicator of FIG. 11;

FIG. 13 is a top view of the gas application device of FIG. 11 as applied to an open surgical site in the thoracic cavity;

FIG. 14 is a perspective view of another embodiment of a movable gas applicator;

FIG. 15 is a perspective view of another embodiment of a movable gas applicator;

FIG. 16A is a top schematic view of a gas application device according to an embodiment having a plurality of feeder tubes;

FIG. 16B is a top view of an embodiment of the gas application device of FIG. 16A;

FIG. 17 is a top view of a diffuser of a gas application device;

FIG. 18 is a cross-sectional view of the diffuser of the gas application device of FIG. 17 taken along the line 18-18;

FIG. 19 is a cross-sectional view of the gas application device of FIG. 16B;

FIG. 20A is an exploded perspective view of the gas application device of FIG. 3;

FIG. 20B is an exploded perspective view of another embodiment of gas application device having a large draping sheet.

### DETAILED DESCRIPTION OF PREFERRED EMBODIMENTS

Gas Application Devices Having Gas Conduits

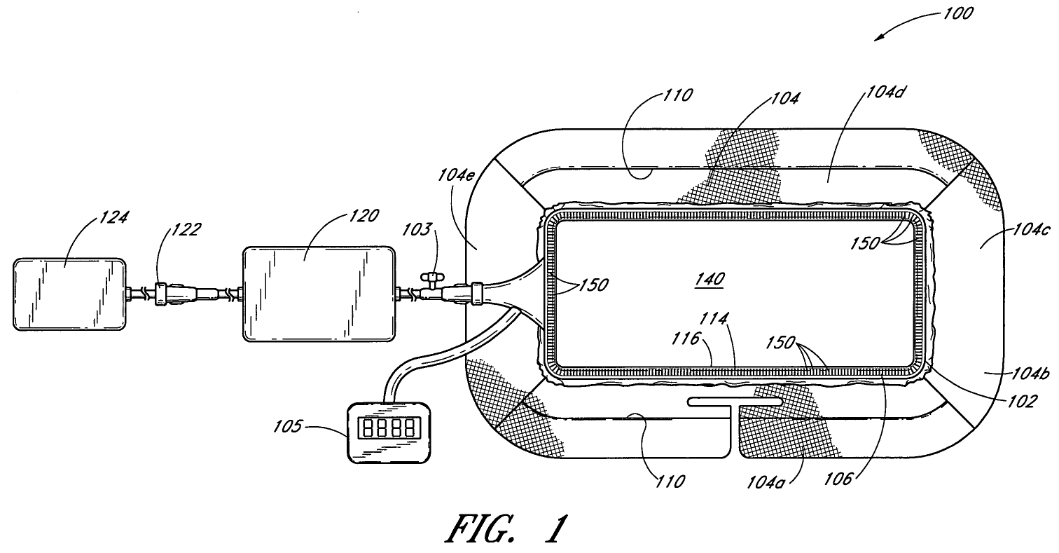

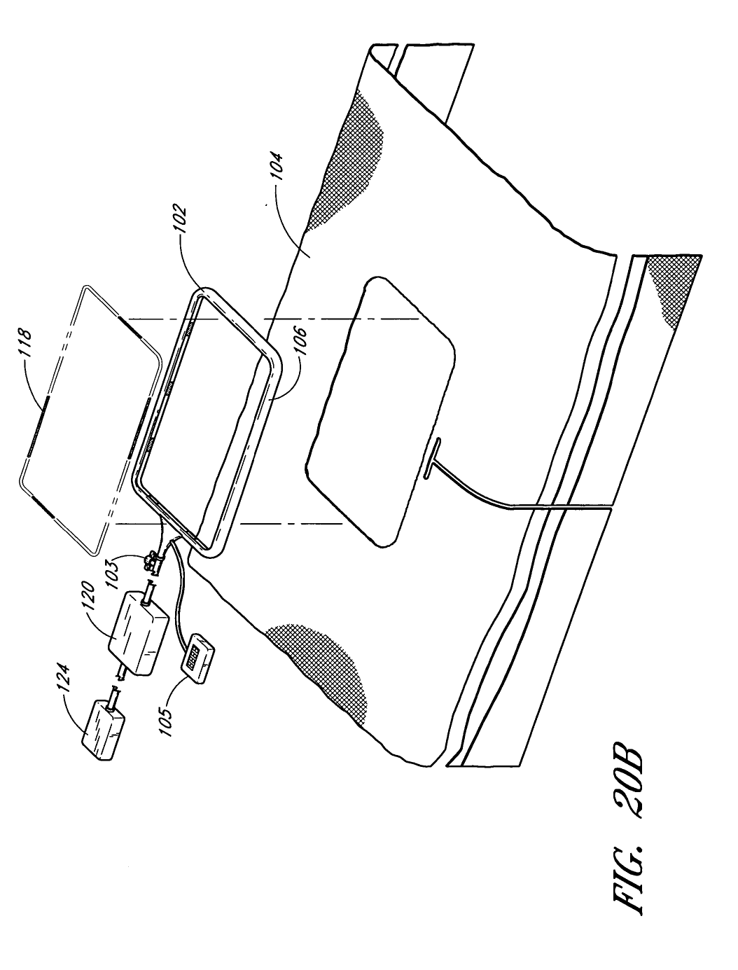

FIGS. 1-7 illustrate a gas application device 100 according to certain embodiments of the present invention. FIG. 20A illustrates an exploded perspective view of the gas application device 100 depicted in FIG. 3. The gas application device comprises a support structure such as a gas conduit 102 and a plurality of gas outlets 202 arranged along the gas conduit 102. Each of the gas outlets 202 is configured to operate at approximately the same volume flow rate. As used herein, the direction upstream is used to refer to a direction along a gas flow path towards a gas source 124 that can be supplying the gas application device 100 with a gas flow. The direction downstream is used to refer to a direction along a gas flow path toward the gas outlets 202.

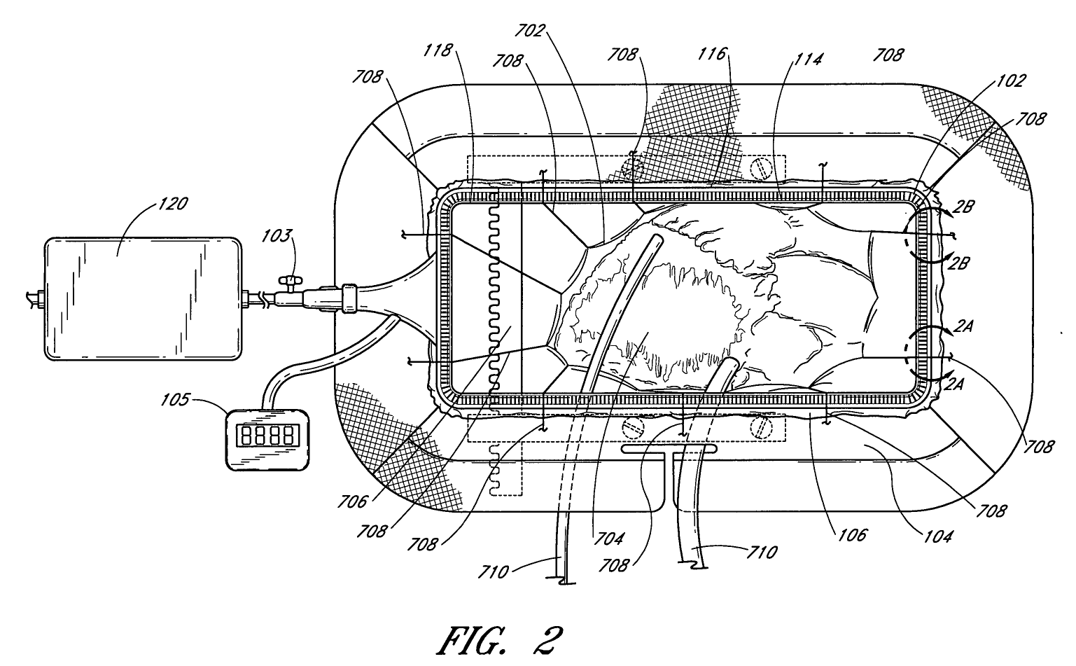

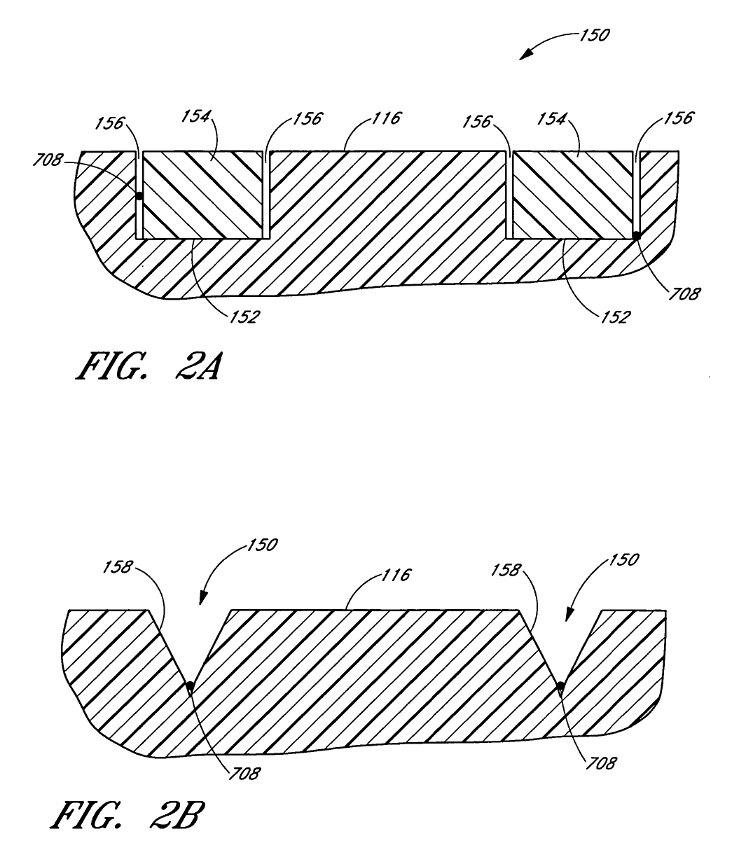

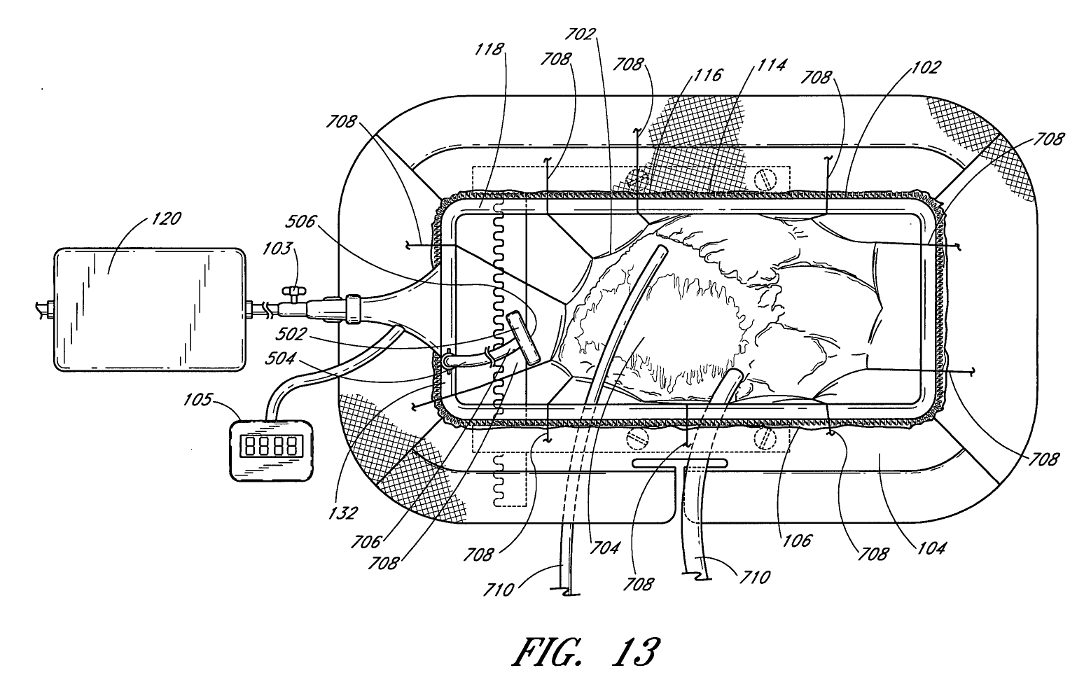

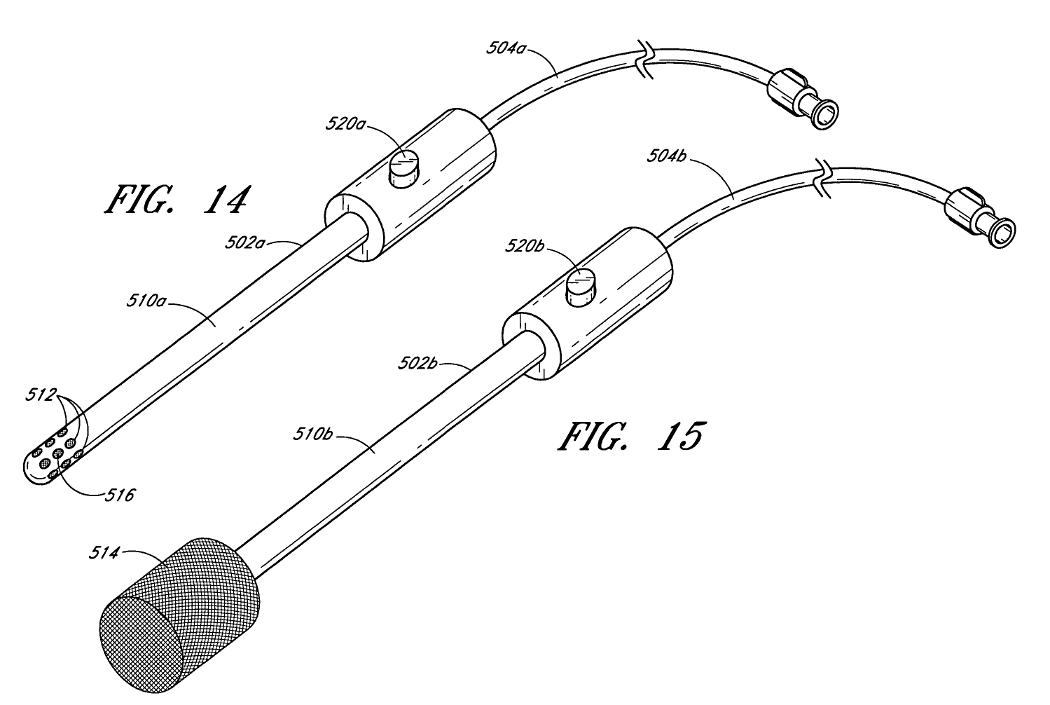

The gas application device 100 is preferably configured to supply carbon dioxide gas to a predetermined surgical site 702 (FIGS. 2 and 13) to displace air from the surgical site 702. The risk of embolism, while pronounced in heart surgeries, is present in other surgeries as well. For example, the risk of embolism is also present in minimally-invasive cardiac surgeries where the vasculature is accessed percutaneously from a point removed from the pericardial cavity, such as through an insertion site overlying a femoral artery. Moreover, the risk of embolism is also present in other open cavity surgeries such as open knee surgeries. In certain embodiments, the gas application device 100 is configured to supply gas to the pericardial cavity for a heart surgical procedure. In other embodiments, the gas application device 100 can be configured to supply gas to other surgical sites.

A surgical site environment of carbon dioxide gas advantageously has embolism reducing and infection inhibiting properties. Carbon dioxide gas has enhanced solubility in blood, thereby reducing risk of gas embolism. Moreover a carbon dioxide gas environment at a surgical site can inhibit growth of certain common infective bacteria such as staphylococcus aureus as compared with a surgical site environment of air. Therefore, the gas application device 100 can include a gas inlet port 122 configured to be in gas communication with a carbon dioxide gas supply. However, it is recognized that the various embodiments of gas application device 100 disclosed herein can be used with a supply of a different gas, including other gases, mixtures of other gases, or mixtures of other gases with carbon dioxide.

The gas flow from the gas supply 124 can be conditioned to a desired state for application to a patient. Before entering the gas application device 100 through the gas inlet port 122, the gas can pass through a conditioning unit 120, or, in some embodiments, one or more gas conditioning features can be provided within the gas application device 100 or in a unit attached to the gas application device 100. The conditioning unit 120 can include one or a combination of a filtration unit, a humidifier, a gas flow metering device, and a temperature modifying device such as a heater or a refrigeration unit. Alternatively, the gas flow could pass through separate devices for one or more of filtration, humidification, metering, and heating. In embodiments where the gas application device 100 is used with a mixture of two or more gases, the conditioning unit 120 could include a gas mixing valve to meter the mixture of gases to a desired ratio. Alternatively, a pre-mixed gas source could be used.

In certain embodiments, the gas application device 100 can be configured to provide flow visualization. Flow visualization advantageously allows a medical professional to visually verify that a local gas-rich atmosphere has been created by the gas application device 100 about the surgical site. The flow visualization can be provided by mixing the gas flow from the gas source 124 with a visualization agent such as a water vapor fog or other colored or non-colored visualization agents. A temperature differential between the gas flowing from the gas application device 100 and the ambient air, maintained by the temperature modifying device of the conditioning unit 120, could be used to maintain water vapor fog in the gas flow. In certain embodiments, the visualization agent can be selectively activated and de-activated such that a medical professional can verify the gas flow by activating the flow visualization, then can deactivate the flow visualization to obtain a clear view of the surgical site. Alternatively, one or more gas detectors can be used with the gas application device 100 to allow the medical professional to monitor the gas content of the local atmosphere at the surgical site. The gas detector can include one or more probes positioned around the gas conduit 102 or extending from the gas conduit 102 into the surgical site. For example, various commercially-available carbon dioxide level detectors can be used in conjunction with a gas application device 100 supplying carbon dioxide.

In certain embodiments, the gas application device 100 includes a flow controller 103 such as a valve to allow a medical professional to selectively control the flow of gas to the gas conduit 102. The flow controller 103 can be a valve that allows an operator to control the flow rate of gas variably between a substantially completely shut off state and a full flow state. The flow controller 103 can be structured in various ways, such as a rotary valve, a push button valve, or other valve structures that allow user selection of flow rate. For example, the flow controller 103 can be a two-state valve, allowing a user to selectively activate or terminate a flow of gas through the gas conduit 102. In some embodiments, the flow controller 103 is positioned downstream of the gas source 124 and the conditioning unit 120 such that a medical professional can manipulate the flow controller 103 during a surgical procedure. In certain embodiments, the flow controller 103 can be adjacent the gas inlet port 122. Advantageously, this positioning of the flow controller enables medical professionals to adjust the gas flow rate to the gas conduit 102 without stepping away from the patient to access a valve on the gas source. Stepping away from the patient to adjust a valve on the gas source 124 can consume precious time during a surgical procedure and can require additional sterility measures, such as sterilizing a valve on the gas source 124 or requiring a medical professional to change gloves or scrub hands after adjusting the valve.

In some embodiments, the gas application device 100 includes a flow rate indicator 105. Various mechanical and electrical displays can be used to indicate the flow rate of the gas in the conduit. For example, a digital display, analog gauge, or mechanical float could be used to indicate the flow to or through the conduit. In certain embodiments, the flow rate indicator 105 can be positioned on the gas conduit 102 itself such that it can be easily viewed by medical professionals conducting a surgical procedure. Alternatively, the flow rate indicator 105 can be positioned upstream of the gas conduit 102. In some embodiments, the flow rate indicator 105 can be integrated with the flow controller 103.

In certain embodiments, the gas application device 100 is intended for use in a single surgical procedure only. Single use only embodiments of gas application device 100 can be packaged for sale in sterilized packages such as sealed plastic pouches. It is contemplated that during a surgical procedure, the gas application device 100 may be soiled with blood, medications, and other fluids. In lieu of cleaning and sterilizing the gas application device 100 for each subsequent use, it can be discarded after the surgical procedure. Moreover, as further discussed below, certain features of the gas application device 100, such as suture holders 114 (FIG. 2) may perform better on a first use than on subsequent uses. Alternatively, in other embodiments, the gas application device 100 can be configured to be sterilized and reused. In these multiple-use embodiments, the gas application device 100 is preferably constructed of a material or materials that can withstand repeated cleaning and sterilization by autoclave or chemical agents.

The Gas Conduit

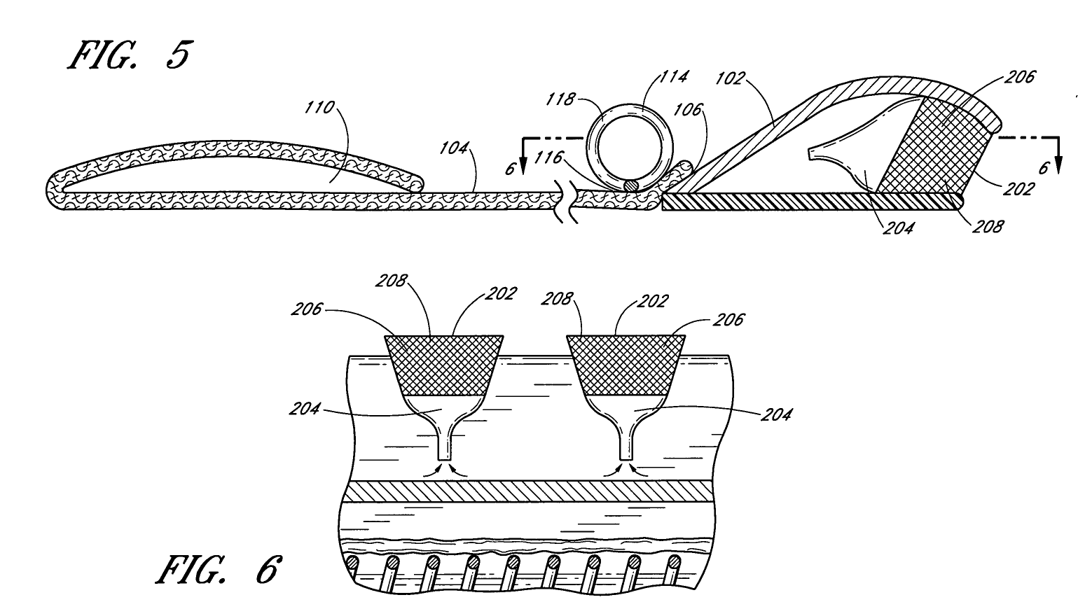

As illustrated, the support structure is a gas conduit 102 comprising a generally hollow tubular segment that allows gas flow therethrough. It will be appreciated by one of skill in the art that in other embodiments of gas irrigator, the support structure could comprise multiple gas conduits, frame members, or other supporting structures, some of which are further discussed below with reference to FIGS. 16A and 16B. To allow the gas conduit 102 to have a low profile over a surgical site, the hollow tubular segment can have a generally tear-shaped cross-sectional profile as illustrated in FIG. 5. Advantageously, this low profile facilitates access to a surgical site. Other cross-sectional profiles that allow sufficient flow but provide a low-profile gas conduit can also be utilized for the gas conduit 102. For example, a generally rectangular profile having a relatively low height and high width can provide a low vertical profile and sufficiently high flow rate. Alternately, if a low profile gas conduit 102 is not desired, a generally cylindrical tubular section having a generally circular cross-sectional profile can be used.

In some embodiments, the gas conduit 102 is pliable. When applied to a pericardial cavity during a heart surgery, a pliable gas conduit 102 can overlie various medical instruments such as surgical retractors and various fluid supply and return conduits such as supply and return tubes for a heart-lung machine. Moreover, the pliability of the gas conduit 102 can enhance the ability of the gas conduit 102 to conform to a contoured surgical site such as a patient's chest. Additionally, in certain embodiments, it is desirable that the gas conduit 102 be sufficiently pliable to be foldable when not in use. For example, it can be desirable that the gas conduit 102 be folded into a reduced area package when packaged for distribution. The gas conduit 102 may be constructed of one of a variety of materials to provide the desired pliability. For example depending on other desired properties, the gas conduit 102 could be constructed of one of a variety of metals, metal alloys, plastics, natural rubbers, and synthetic rubbers.

In certain embodiments, the gas conduit 102 may comprise a composite structure of a plurality of materials. For example, the gas conduit 102 can comprise a flexible plastic generally hollow tubular segment ring that is integrated with a pliable metal wire support structure. This combined plastic and metal construction advantageously provides shape adaptability as the gas conduit 102 could be bent and twisted into a desired shape, then the wire support structure can maintain the desired shape. For example, the gas conduit 102 could be bent to closely fit over the contours of a specific patient's chest. In other embodiments, the gas conduit 102 can comprise a flexible generally hollow tube segment that is integrated with a pliable metal wire support structure comprising a metal material having shape memory characteristics. Thus, the gas conduit 102 could be bent to a desired shape such as a patient's chest, but returned to the initial shape as desired.

In the illustrated embodiments, the gas conduit 102 is shaped to overlie a predetermined surgical site 702 (FIGS. 2 and 13). The gas conduit 102 can be formed into a loop having a generally rectangular shape with rounded comers. In the illustrated embodiments, the loop shape 132 is sized to overlie a pericardial cavity 704 during a heart surgical procedure. The gas conduit loop 132 bounds an interior area 140 that is left open over the surgical site 702 for substantially unobstructed access to the surgical site 702. Different sizes of the gas conduit loop 132 can be provided to facilitate application of the gas conduit 102 to patients of differing sizes. Other shapes and configurations of the gas conduit 102 are possible to overlie other surgical sites and are considered within the scope of the present inventions. For example, a gas conduit 102 formed as a circular ring can be beneficial for use at certain surgical sites, a generally L-shaped gas conduit 102 can be beneficial for use at other surgical sites, and a generally straight gas conduit 102 can be beneficial for use at still other surgical sites.

Gas Outlets

A plurality of gas outlets 202 (FIG. 6) each provide an exit for the gas flow through the gas conduit 102. As illustrated in FIGS. 5 and 6, the gas outlets 202 are disposed on an inner edge of the gas conduit loop 132 such that gas exiting the gas conduit 102 through the outlets 202 is directed towards the interior area 140 generally bounded by the gas conduit 102. Preferably, the gas outlets 202 are substantially evenly spaced along the gas conduit 102. While this interior-facing gas outlet 202 configuration provides beneficial gas application to a pericardial cavity 704, other arrangements of the gas outlets 202 can be made to configure the gas application device 100 to a specific surgical site 702.

In some embodiments, the gas outlets 202 can be movably coupled to the gas conduit 102. For example, the gas outlet 202 can be pivotally coupled to the gas conduit 102 about one or more axes such that a medical professional can orient the gas outlets in a desired direction. One, some, or all of the gas outlets 202 of a gas application device 100 could be pivotally coupled to provide directional adjustability. This pivotal coupling can be provided by various structures such as a mechanical pivot such as a pinned connection, or by selection of a flexible material in an interface between the gas conduit 102 and a gas outlet 202. Advantageously, this directional adjustability can allow a gas application device 100 to be adjusted to provide substantially uniform gas coverage even when applied to body cavities having unusual or extreme contours such as when the gas application device is applied in a knee or hip procedure.

Flow Limiters

A plurality of flow limiters 204 can be positioned within the gas conduit 102 upstream from the gas outlets 202. FIGS. 5 and 6 depict cross-sectional views of flow limiters. FIG. 5 illustrates a side view of a flow limiter 204 within the gas conduit. FIG. 6 illustrates a cross-sectional top view of two adjacent flow limiters 204. Each of the plurality of flow limiters 204 is fluidly coupled to a corresponding gas outlet 202. If no flow limiters 204 were present in a loop-shaped gas application device, gas flowing through the gas conduit 102 would tend to exit the outlet 202 or outlets 202 nearest the gas inlet port 122. This tendency of gas to exit the gas conduit 102 out of a particular gas outlet 202, if not addressed, would result in an uneven distribution of the gas at the surgical site. The flow limiters 204 can be effectively used to address this uneven distribution. By configuring the flow limiters 204 such that gas outlets 202 near the gas inlet port 122 are more resistant to flow than gas outlets 202 farther downstream from the gas inlet port 122, the uneven distribution can be addressed. Preferably, the flow resistance of the flow limiters 204 will be varied such that each of the gas outlets 202 is configured to operate at approximately the same volume flow rate. Thus, the gas outlets 202 evenly distribute the gas flow in the surgical site. Advantageously, this even distribution of gas flow reduces the occurrence of local air pockets and eddies that was prevalent with the prior art gas application devices and that increased the risk of air embolism in those devices.

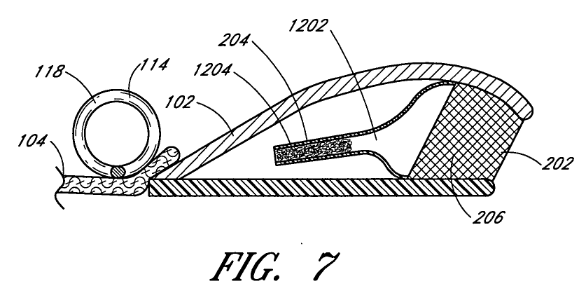

In various embodiments, the flow limiters 204 each comprise a generally tubular body 1202 that is at least partially filled by a gas-permeable foam material 1204 (FIG. 7). A cross-sectional view of a flow limiter 204 having a gas-permeable foam filling is depicted in FIG. 7. The gas permeable foam material 1204 has voids in the foam that allow the passage of gas, but restrict the ability of gas to flow freely through the material. Flow limiters 204 having relatively high resistance to flow can be achieved by increasing the density (that is, reducing the void space) of the gas permeable foam material 1204. Flow limiters 204 having relatively high resistance to gas flow can be achieved by adding more gas permeable foam material 1204 than is present in flow limiters 204 having a relatively lower resistance to gas flow, or by using different types of foams with differing gas-flow properties in the flow limiters 204. The flow limiters 204 can be structured in many different ways. For example, differences in the interior cross-sectional areas of the flow limiters 204, or smaller diameter internal tubing and/or contouring can also serve to vary the flow-impeding properties of the flow limiters 204. As discussed above, these flow limiters 204 having a relatively high resistance to flow can be located near a gas inlet port 122 to provide a substantially uniform flow distribution circumferentially around the surgical site.

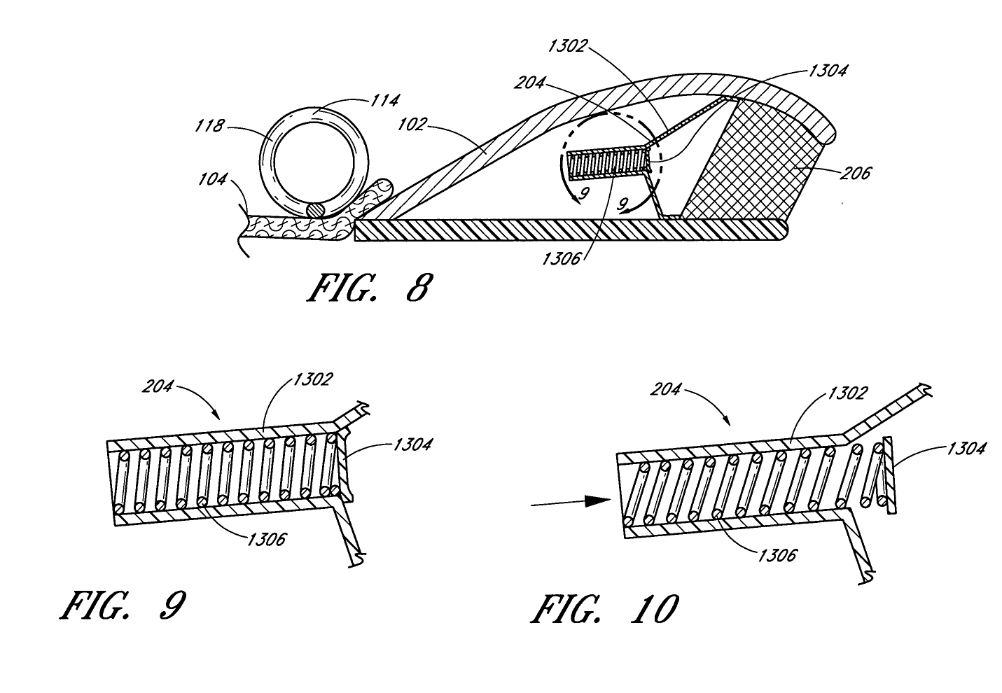

The flow-limiting finction can also be achieved mechanically. For example, FIGS. 8-10 depict a spring-based flow limiter 204. FIG. 8 illustrates a cross-sectional view of a flow limiter 204 in the gas conduit 102 of a gas application device. Spring-based flow limiters 204 each comprise a generally tubular body 1302, an obstructing element 1304, and a spring 1306. The obstructing element 1304 is movable between an open position wherein the generally tubular body 1302 admits gas flow and a closed position wherein the generally tubular body 1302 rejects gas flow. The spring 1306 operatively connects the obstructing element 1304 to the generally tubular body 1302. The spring 1306 is configured to bias the obstructing element 1304 into the closed position. FIG. 9 illustrates the closed position of the obstructing element 1304 with respect to the generally tubular body 1302, and FIG. 10 illustrates the open position of the obstructing element 1304. The spring can be configured such that a predetermined gas flow pressure through the flow limiter 204 moves the obstructing element 1304 towards the open position. Flow limiters 204 having relatively high resistance to gas flow therethrough (for use near the gas inlet port 122) can include stiffer springs 1306, or shorter springs 1306 than flow limiters 204 having lower resistance to gas flow therethrough. Many other types of spring-based and other mechanical flow limiters are possible.

Diffusers

In various embodiments, gas application devices can include a plurality of diffusers 206. Each of the plurality of diffusers 206 are preferably disposed within the gas conduit 102 and fluidly coupled to a corresponding one of the plurality of flow limiters 204. The diffusers 206 are positioned downstream from the flow limiters 204 such that a downstream end of the diffusers 206 defines a gas flow exit from the gas outlet 202. The diffusers are comprised of a diffusion material 208 capable of diffusing a gas flow into tiny microcurrents. For example, the diffusers 206 can be comprised of a diffusion material 208 composed of microfiber, airstone, flexible foam, rigid foam, polyurethane foam, other fluid resistant foam, or other materials having gas diffusive properties. Advantageously, this diffusion of the gas flow into microcurrents presents a surgical site with a generally uniform mixed volume of gas, whereas prior art gas application systems often resulted in local currents, eddies, and air pockets. The uniform layer of gas, created through the use of diffusers advantageously displaces air from a surgical site and thus reduces the risk of embolism from unwanted air entrained into the bloodstream at a surgical site.

Draping Sheet

Additionally, certain embodiments of the gas application device can include a draping sheet 104. Advantageously, the draping sheet 104 can cover tools and tubings near a surgical site that can otherwise become entanglement sites for sutures if not covered. The draping sheet 104 can be comprised of absorbent material such as towel-like material. The absorbent material forming the draping sheet 104 tends to contain any splatters of blood, medication, antiseptic, etc. to an area adjacent the surgical site. Alternately, the draping sheet 104 can be fluid resistant. It is contemplated that draping sheet 104 can be constructed of one of a variety of natural or synthetic sheeting materials. Desirably, the sheeting material of the draping sheet 104 exhibits a cloth-like texture and fluid resistance. In embodiments where the draping sheet 104 is fluid resistant, the draping sheet can include a pouch 110 (FIGS. 1, 3, 5) positioned around the periphery of he draping sheet 104 to catch fluids that land on the draping sheet 104. Desirably, the pouch 110 can be comprised of or lined with an absorbent material such that fluids that run off to the pouch are retained in the pouch.

The draping sheet 104 preferably has a weight and thickness that allows the draping sheet 104 to remain substantially in position once it has been located over a surgical site. Additionally, the weight of the draping sheet 104 allows it to conform substantially to contours of the patient's body underlying the draping sheet. This conformity of the draping sheet 104 with the patient's body creates a substantially enclosed pocket to retain gas from the gas application device 100. Advantageously, this retention of gas reduces the incidence of gas escaping from the surgical site, thus reducing the gas flow rate required to maintain a local gas-rich atmosphere around the surgical site.

The draping sheet 104 may be sized to correspond to a particular surgical site application. In certain embodiments where the gas application device 100 is intended for use over the pericardial cavity in a heart procedure, for example, the draping sheet 104 can be sized to drape over the patient's chest, over the operating room bed, and downward towards the floor from the bed in a width dimension. Alternatively, the draping sheet 104 could be sized to drape over substantially all of the patient's chest, drape over a portion of the patient's chest, or drape over an area of the patient's chest around the surgical site. FIG. 20A illustrates a relatively small 104 draping sheet configured to cover an area adjacent a surgical site. FIG. 20B illustrates a relatively large draping sheet 104 configured to substantially cover at least a portion of a patient's body. In a length dimension, the draping sheet 104 could be sized to drape over the patient from substantially the patient's neck region to substantially the patient's abdominal region. Alternatively, the draping sheet 104 could be sized to cover more or less of the patient in a length dimension. It is contemplated that draping sheets intended for use at other surgical sites can have different dimensioned configurations.

Desirably, the draping sheet has a color that provides contrast against sutures used in surgical procedures. Preferably, the draping sheet will be of a color that provides contrast against blue, white, or black sutures as are commonly used in cardiac procedures. In certain embodiments, a gray draping sheet 104 can provide the desired color contrast.

The draping sheet is connected to the gas conduit 102 about an outer edge 106 of the gas conduit 102. The draping sheet 104 can be removably attached to the gas conduit 102 such as by hook and loop fasteners so that a soiled draping sheet 104 can be exchanged for a clean one, or a draping sheet 104 can be exchanged for one of a different size. In certain embodiments, the draping sheet 104 at least partially seals about the periphery of a surgical site providing a barrier to escape of a heavier-than-air gas such as carbon dioxide from within the surgical site. Advantageously, this draping sheet 104 is integrated with the gas application device 100 and is thus easily applied to a surgical site. In contrast, known draping materials, which generally consist of individual towels, can be individually hand placed in a desired location near the surgical site, leaving a field exposed for the actual surgical site. Additionally, the extended surface area presented by the draping sheet 104 enhances the stability of the gas application device 100 when it is positioned at a surgical site.

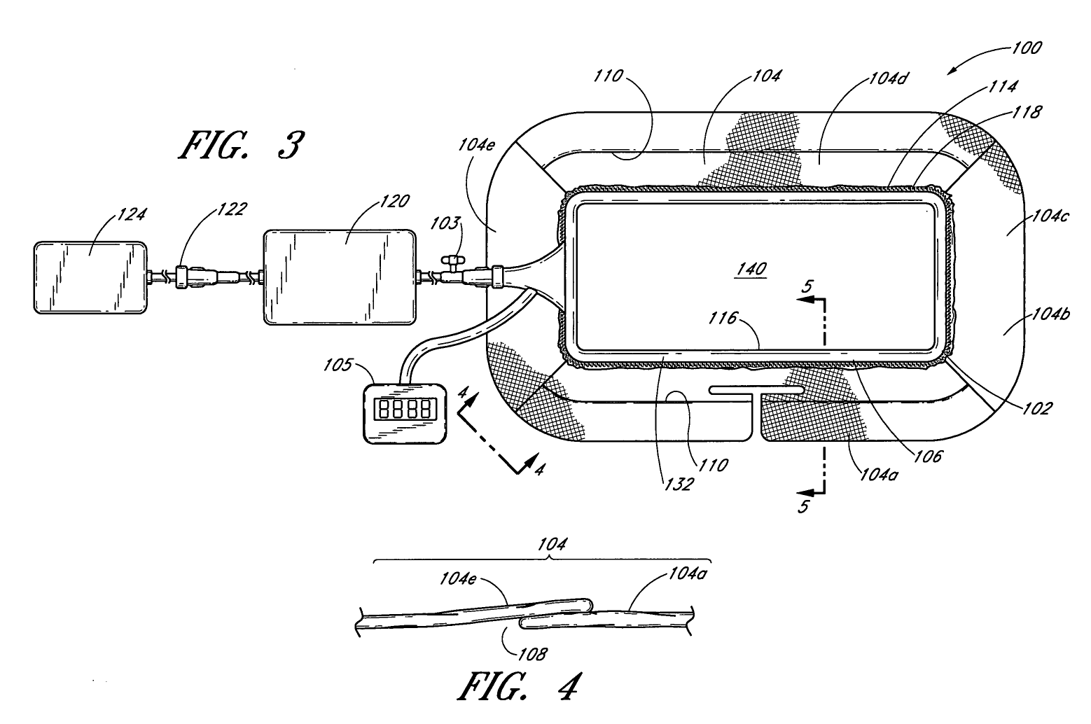

The draping sheet 104 can be composed of a plurality of draping sheet segments 104a-e. As shown in FIG. 4, one draping sheet segment, 104e can overlap an adjacent draping sheet segment 104a such that a clearance 108 is created under the draping sheet segment. This clearance 108 can allow the placement of access tubes, wires, or other medical implements at the surgical site 702. For example, as illustrated in FIGS. 2 and 13, inflow and outflow conduits 710 connecting a heart with a heart-lung machine can be passed through the clearance 108 under the gas application device 100. Advantageously, this segmented, overlapping construction facilitates placement and adjustment of underlying equipment during different phases of a surgical procedure.

In some embodiments, when a gas application device is configured to be placed over an open pericardial cavity, the draping sheet 104 is segmented into 5 individual draping sheet segments: two upper draping sheet segments 104b, 104c, one lower draping sheet segment 104e, one left draping sheet segment 104a, and one right draping sheet segment 104d (FIGS. 1, 3). The top side of the draping sheet 104 is formed of two segments 104b, 104c to accommodate placement of a large clamp (not shown) commonly used to occlude the aorta of a patient during surgery. In other embodiments, the top side of the draping sheet can be constructed of a single panel with a central split such that the draping sheet 104 is formed of 4 individual draping sheet segments. Preferably, the left draping sheet panel 104a (with reference to a patient's left side) includes a cut out segment to allow for the placement of a retractor system commonly used during mitral valve surgeries. Alternately, the left draping sheet panel can be removably attached to the gas conduit 102, such as with hook and loop fasteners to allow placement of the retractor system. While certain slots and recesses in the draping sheet 104 are described with reference to surgical instruments commonly used in heart surgeries, it is contemplated that in other embodiments, a draping sheet 104 can have slots or recesses at different orientations and locations than those described. According to certain embodiments, the draping sheet 104 has slots and recesses to accommodate the usage of surgical instruments and equipment anticipated to be used at a predetermined surgical site.

Additionally, as previously described, a pouch 110 (FIGS. 1, 3, 5) can be integrated into the gas application device of the present invention. The pouch 110 can be formed of an upper surface 112 of the draping sheet 104. The pouch 110 provides easily accessible storage of surgical tools during a surgery. Advantageously, the pouch 110 can be configured to catch dropped tools and liquid spills and splatters, or to temporarily store tools placed therein during a surgical procedure. Additionally, as discussed above, the pouch 110 can comprise an absorbent inner layer such that liquid running off of the draping sheet 104 is absorbed and retained in the pouch. In addition to or in place of a pouch 110 at the periphery of one or more sides of the draping sheet 104, in various embodiments, the draping sheet can include one or more pouches configured to accommodate surgical instruments and equipment anticipated to be used at a predetermined surgical site.

Suture Holders

Certain embodiments of the gas application device 100 further comprise a plurality of suture holders 114 located on an upper surface 116 of the gas conduit 102. The plurality of suture holders 114 can comprise a plurality of substantially evenly spaced grooves 150 (FIGS. 1 and 2) formed in the upper surface 116 of the gas conduit 102. In other embodiments, the suture holders 114 can be configured for a particular surgical procedure: a predetermined number of grooves 150 can be located at predetermined positions on the upper surface 116 of the gas conduit 102. For example, in some types of heart procedures such as valve replacement surgeries it may be required to anchor approximately 15-20 suture pairs. A gas application device 100 configured for use in a heart valve replacement surgery, therefore, can have a desired number of grooves positioned to retain these 15-20 suture pairs. The grooves 150 are configured to hold tension on sutures placed in them. FIG. 2 illustrates sutures 708 held in place by suture holder grooves 150. The grooves 150 allow a suture to be placed therein transversely to a longitudinal axis of a groove 150. The suture can then be removed by transverse extraction from the groove 150. But the grooves 150 can hold tension on a suture placed therein in a direction generally parallel to the longitudinal axis of the groove 150.

Various configurations of grooves are contemplated in various embodiments of the gas application device 100. Several different embodiments are illustrated in FIGS. 2A and 2B. FIG. 2A illustrates embodiments of suture holder wherein each groove 150 comprises a recess 152 in the upper surface 116 of the gas conduit 102 perferably and a post 154 disposed in the recess 152. The upper surface 116 of the gas conduit 102 abuts the post 154 at an interface 156 on each side of the post 154. A suture 708 can be inserted into the interface 156, to be retained by clamping forces between the post 154 and an adjacent wall of the recess 152. The posts 154 and the upper surface 116 of the gas conduit 102 are desirably formed of materials chosen such that they allow easy insertion and removal of sutures and exhibit sufficient clamping forces to retain sutures. For example, in various embodiments, the upper surface 116 of the gas conduit 102 includes a layer of a relatively dense rubber into which a plurality of recesses 152 is cut, molded, or otherwise formed, and posts 154 formed of hard plastic are adhered to a lower surface of the recesses. In some embodiments, the posts 154 comprise materials that are colored to contrast with the a color of the upper surface 116 of the gas conduit 102. Advantageously, this color contrast assists in the location of the suture holders 114 by a medical professional.

With reference to FIG. 2B, in other embodiments, the grooves 150 comprise substantially v-shaped recesses 158 formed by molding, cutting, or some other technique, in a rubber layer disposed on an upper surface 116 of the gas conduit 102. Each v-shaped recess 158 has two convergent sidewalls. Sutures 708 can be inserted in to the v-shaped recesses 158 and pulled downward into a narrow end of the v-shape where the side walls of the v exert clamping force on the suture 708. The rubber layer is desirably formed of a relatively dense rubber that allows insertion and removal of the sutures, but maintains clamping force on an inserted suture. The rubber layer can be of a relatively small thickness, for example, about ¼ cm, to maintain the low profile of the gas conduit 102.

In some embodiments, the plurality of suture holders 114 can comprise a coil spring 118, affixed to the upper surface 116 of the gas conduit 102 (FIGS. 3, 5, 11, 13, 20A, and 20B). One coil spring 118 loop can extend around the perimeter of the gas conduit 102 (FIGS. 3, 5, 11, and 13), or a plurality of coil spring 118 segments can be positioned at spaced apart locations around the perimeter of the gas conduit (FIGS. 20A and 20B). Sutures are placed between adjacent turns of the coil spring 118, and friction between the adjacent turns of the coil spring 118 holds tension on the sutures. Preferably, the coil spring 118 is positioned at a narrow side of a generally tear-shaped gas conduit 102 to maintain the low profile of the gas conduit. Also, preferably, the coil spring 118 is sized with an outer diameter sufficiently small so that it does not increase the overall profile depth of the gas applicator device 100.

In other embodiments, a mechanical suture holder such as a cam-like lever arm can be used to retain each suture or suture pair. Various commercially available mechanical suture holders can be integrated with the gas application device 100. For example, cam-type suture holders as are typically used in conjunction with a cardiac positioning system such as the Medtronic Octopus® system could be coupled to an upper surface of the gas conduit 102 or the draping sheet 104 to retain sutures.

Movable Gas Applicator

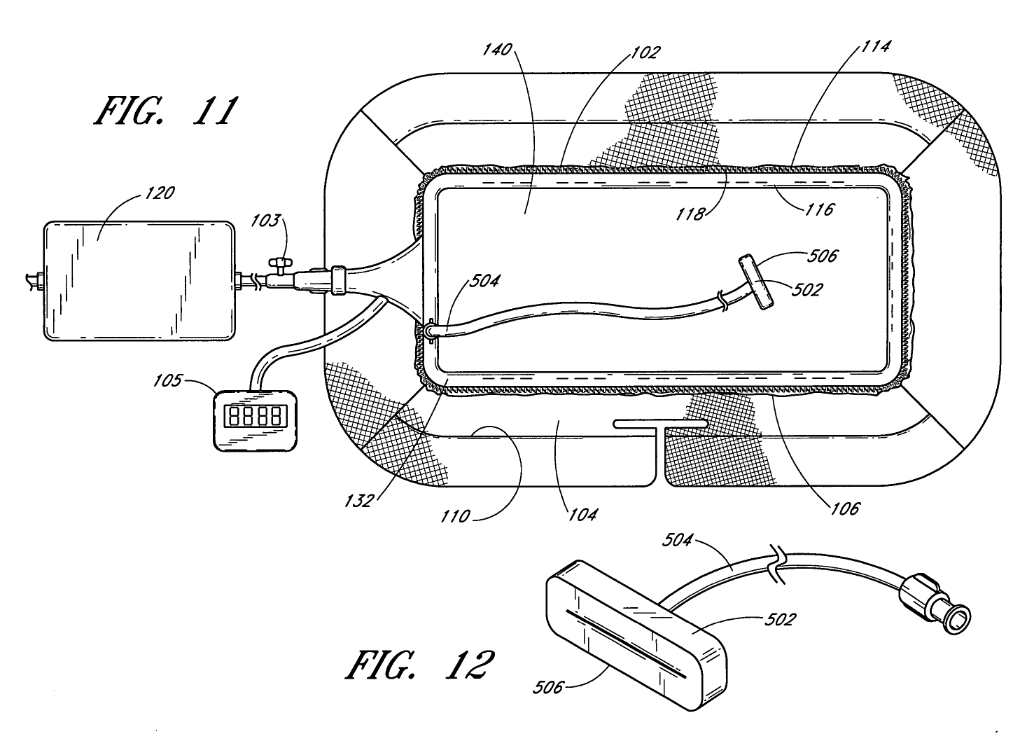

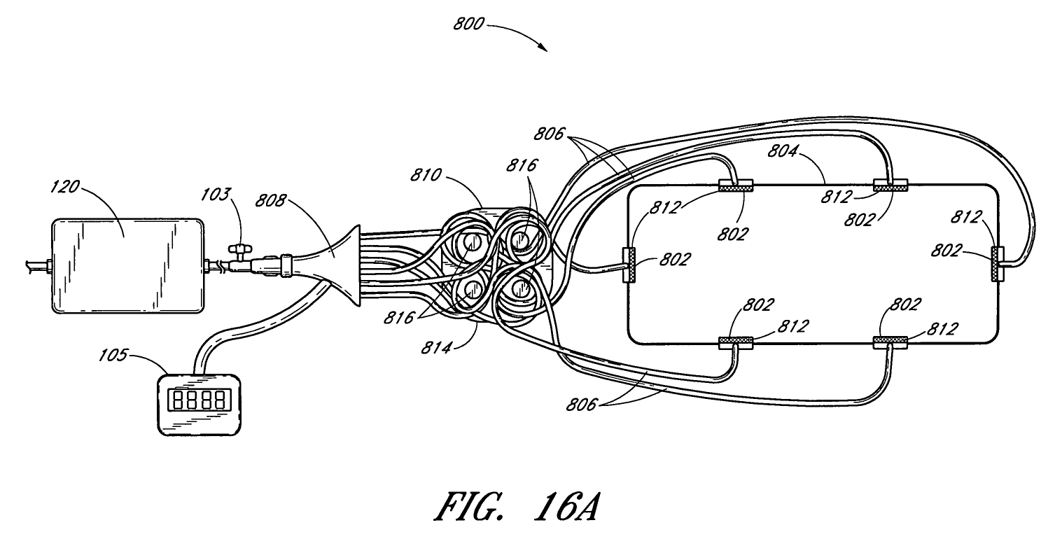

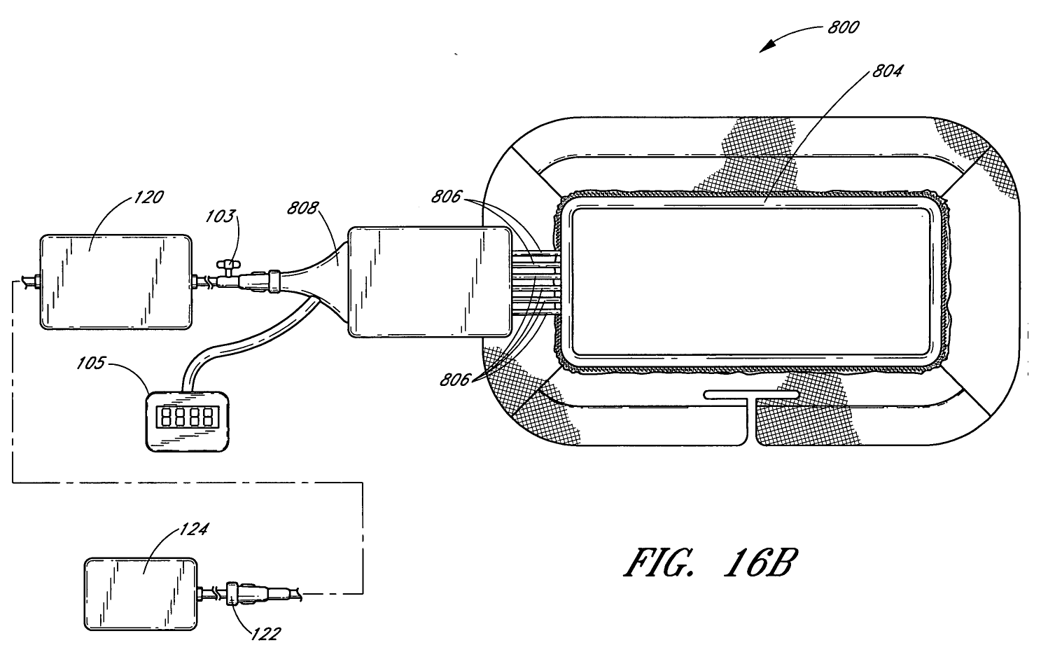

FIGS. 11-15 depict various embodiments of a gas application device and a movable gas applicator 502. FIG. 11 depicts a gas application device with the movable gas applicator 502 fluidly coupled to the gas conduit 102. FIG. 12 depicts a perspective view of the movable gas applicator 502. FIG. 13 depicts the gas application device of FIG. 11 as applied to the pericardial cavity 704. FIG. 14 depicts an alternate movable gas applicator 502a embodiment. FIG. 15 depicts a second alternate movable gas applicator 502b embodiment. The movable gas applicator 502 is fluidly coupled to the gas conduit 102 and is removably attachable to the gas conduit 102 at a port. Desirably, the movable gas applicator 502 includes a variable flow controller such as a rotary valve, push button valve, or trigger control 520a, 520b (FIGS. 14 and 15). In some embodiments, the movable gas applicator 502 is fluidly coupled to the gas conduit 102 at a location upstream of the flow controller of the gas conduit 102 such that flow through the movable gas applicator 502 can be selectively activated, adjusted, and terminated independently of the gas flow of the gas conduit 102. In other embodiments, the movable gas applicator 502 is fluidly coupled to the gas conduit at a location downstream of the flow controller for the gas conduit 102. In these embodiments gas must be flowing through the gas conduit 102 for gas to flow through the movable gas applicator 502.

The movable gas applicator 502 has a gas outlet 506 and can include a diffuser to present a diffuse gas flow. The movable gas applicator 502 is fluidly coupled to the gas conduit 102 through a length of tubing 504 such that a surgeon can position the movable gas applicator where a flow of gas is desired in a surgical site 702. Advantageously, this freedom of movement allows a surgeon to ensure that air has been displaced from a surgical site 702 by moving the movable gas applicator 502, with gas flowing out of its gas outlet 506, throughout the surgical site. Also, while the gas conduit 102 generally remains at wound level, the movable gas applicator 502 can be positioned below wound level. Advantageously, this placement allows temporary local gas flows below wound level without permanently obstructing access to the surgical site 702.

Various embodiments of movable applicator can be used to provide a localized flow of carbon dioxide. Two such embodiments are depicted in FIGS. 14 and 15. FIG. 14 illustrates a movable applicator 502a comprising a handheld wand 510a, a plurality of gas outlet holes 512, an integrated diffuser 516, and a trigger control 520a. The handheld wand is preferably connected to the gas source 124 (FIG. 1) distal of any conditioning unit 120 (FIG. 1) through a length of tubing 504a. The connection to the gas source can be to a port on the gas conduit 102. The trigger control 520a allows a surgeon to selectively control the flow of gas from the movable applicator 502a. FIG. 15 illustrates another embodiment of the movable applicator 502b having a handheld wand 510b, a diffuser tip 514, and a trigger control 520b. This movable applicator 502b is connected to the gas source 124 distal any filtration device 120. The trigger control 520b allows a surgeon to selectively control the flow of gas from the movable applicator 502b. The diffuser tip 514 can be a polyurethane foam diffuser material or other suitable material for providing diffuse gas flow. The movable applicator 502, 502a, 502b can be coiled off to remain clear of the surgical site when not in use. The movable applicators 502, 502a, 502b allow a surgeon to spot apply a gas flow to a pocket that is suspected of retaining air. The movable gas applicator can be particularly advantageous in providing deficit carbon dioxide coverage in thin patients, whose pericardial cavity depth can not provide an adequate carbon dioxide environment. For these patients, a spot application of diffused carbon dioxide from a movable applicator 502 can very effectively provide carbon dioxide to the surgical site.

Gas Application Device Having Equal Length Feeder Tubes

In other embodiments of gas application device, substantially equal inner diameter and equal length feeder tubes can be used instead of flow limiters (described above with respect to FIGS. 5-10) to promote a substantially uniform flow of gas around the periphery of a loop-shaped gas conduit. FIGS. 16-19 depict various aspects of gas application devices having substantially equal length feeder tubes.

FIG. 16A depicts a top view of a gas application device 800, the device comprising a plurality of gas outlets 802 disposed on a gas applicator structure 804, each of the gas outlets 802 fed by one of a plurality of feeder tubes 806. The gas application device 800 further comprises a gas inlet port 808. In certain embodiments, the gas application device 800 can comprise a feeder tube routing segment 810. The gas application device 800 illustrated in FIG. 16A includes six gas outlets 802, although it is recognized that more or fewer gas outlets 802 can be present.

The gas inlet port 808 is configured to accept an incoming stream of gas and distribute the incoming stream substantially evenly among the plurality of feeder tubes 806. Any of a number of fluid flow dividers known in the art can be employed in the gas inlet port 808.

Gas Applicator Structure

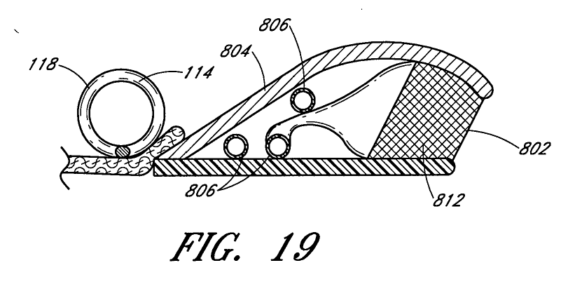

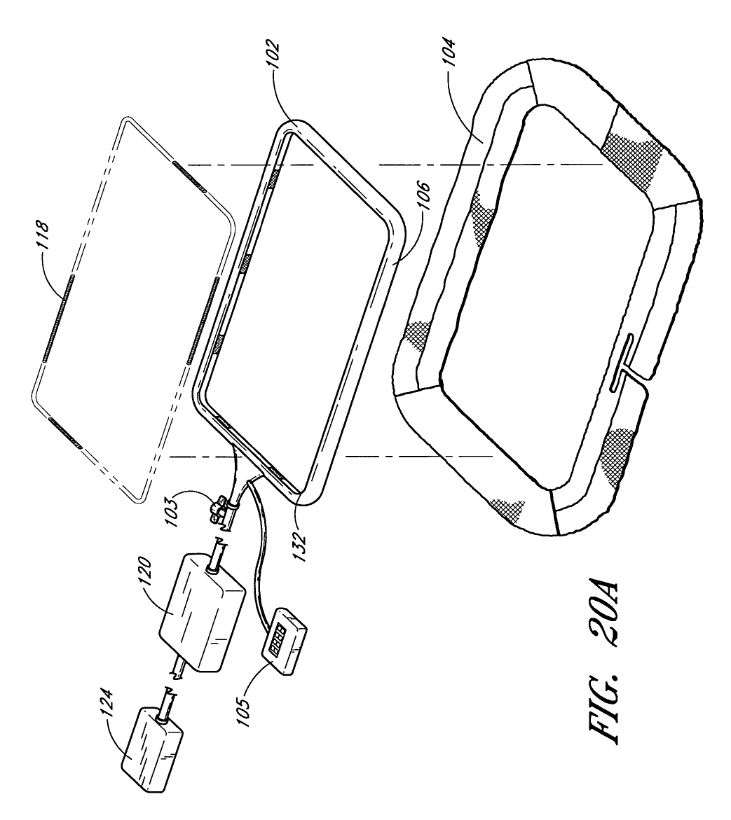

The gas applicator structure 804 is depicted in FIG. 16A in schematic view. It is recognized that the gas applicator structure 804 can be a rigid or semi rigid frame to which the plurality of gas outlets 802 are mounted. Alternately, the gas applicator structure can be a flexible frame such as a pliable wire structure such that the spacing and orientation of individual gas outlets 802 can be shifted relative to the other gas outlets 802 by bending the pliable wire. Alternately, as depicted in FIG. 16B, the gas applicator structure 804 can be a hollow tubular body having a profile similar to the profile of the gas conduit 102 discussed with respect to the gas application device embodiments depicted in FIGS. 1-7. FIG. 19 depicts a cross-sectional view of a gas applicator structure 804 comprised of a hollow tubular body as is depicted in FIG. 16B. Where the gas applicator structure 804 is a hollow tubular body, the feeder tubes 806 can be routed within the hollow tubular body, as shown in FIG. 19.

Gas Outlets

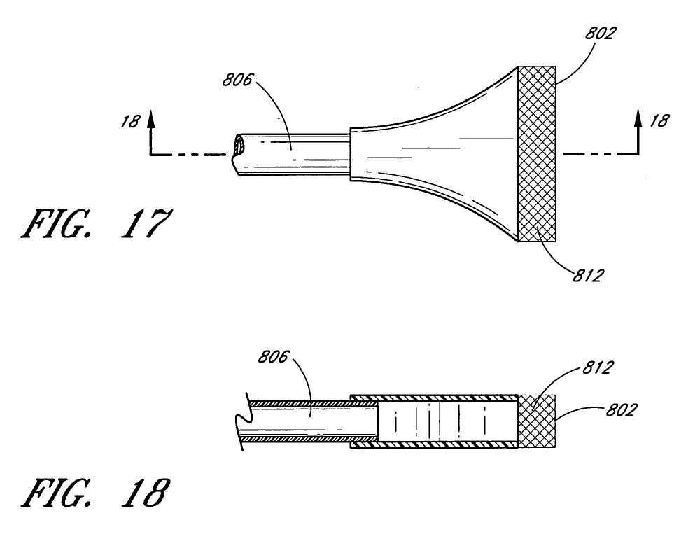

FIGS. 17 and 18 illustrate gas outlets 802 used in the embodiment of FIG. 16A. FIG. 17 is a top view of a gas outlet 802, and FIG. 18 is a cross-sectional view taken along line 18-18 of FIG. 17. Due to the substantially equal lengths of the feeder tubes 806, gas flow will be at substantially equal volumetric flow rates out of each of the plurality of gas outlets 802. Therefore, as shown in FIG. 18, flow limiters are not required to create a uniform circumferential gas flow in a gas application device if the feeder tubes are of substantially equal lengths. Each gas outlet 802 can include a diffuser 812, such as is described above with respect to FIGS. 5 and 6, to create microcurrents in gas flow exiting from the gas outlets 802. As described above with respect to gas outlets 202 in other embodiments of gas application device 100, one, some, or all of the gas outlets 802 can be movably coupled to the gas applicator structure 804 to provide directional adjustability of the gas application device.

Feeder Tube Routing Segment

In certain embodiments, a gas application device including a plurality of substantially equal length feeder tubes, as depicted in FIG. 16A, can further comprise a feeder tube routing segment 810. The feeder tube routing segment 810, can comprise a plate section 814 having a plurality of posts 816 extending from the plate section 814. The feeder tube routing segment 810 can be enclosed in a housing, as depicted in FIG. 16B to reduce the risk that one of the feeder tubes can become snagged on a patient or a surgical instrument, or some other item. Excess lengths of feeder tube 806 can be wrapped around the posts 816. It is recognized that the wrapping of the feeder tubes 806 around the posts, especially if done with tight radius bends, can result in pressure losses in a tightly wrapped feeder tube 806. In order to provide substantially equal volumetric flow out of all of the gas outlets 802, the relative lengths of the feeder tubes 806 can be adjusted. Therefore, while all of the feeder tubes 806 are substantially equal in length, some variation can exist in order to provide substantially equal volumetric flow rates out of all of the gas outlets 802.

Method of Applying Gas to a Surgical Site

A method of applying gas to a surgical site is also provided in various embodiments of the present invention. The method comprises the steps of providing a gas application device, positioning the gas application device proximate the surgical site, and establishing a flow of gas to the gas conduit such that the flow of gas exits the gas outlets.

The gas application device comprises a gas conduit and a plurality of gas outlets arranged along the gas conduit and wherein each of the gas outlets is configured to operate at approximately a same volume flow rate as each other of the plurality of gas outlets. Therefore, advantageously, the gas application device provides a substantially uniform flow of gas about the surgical site. The gas application device can further comprise a plurality of diffusers, each fluidly coupled to one of the gas outlets such that each of the gas outlets expels a diffuse flow of gas consisting of microcurrents.

The gas application device can be positioned proximate a surgical site by placing the gas application device at wound level on a patient's skin adjacent the surgical site. Preferably, the gas conduit of the gas application device is shaped and configured to overlie a particular surgical site, but allow a medical professional access to the surgical site. For example, as depicted in FIGS. 2 and 13, the gas application device 100 is shaped and configured as a loop 132 to overlie a pericardial cavity 704 but allow a medical professional access to the pericardial cavity 704 through the interior area 140 of the gas conduit loop 132.

A flow of gas to the gas conduit can be established by connecting an inlet port of the gas application device to a gas supply, and initiating gas flow from the gas supply. The flow of gas from the gas supply can be humidified, filtered, or both, by fluidly coupling a humidifier and filter to the gas supply upstream of the gas conduit.

In certain embodiments of the gas application method, a movable gas applicator is fluidly coupled to the gas conduit, and the method further comprises the step of positioning the movable gas applicator at a desired location proximate the surgical site. The movable gas applicator is fluidly coupled to the gas conduit by a tube, and the movable gas applicator is configured to be easily positioned and repositioned within a surgical site by a medical professional. Thus, while the gas application device can remain at wound level, the gas applicator can be movably positioned to locally apply gas flow directly to the surgical site below wound level.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. Further, the various features of these inventions can be used alone, or in combination with other features of these inventions other than as expressly described above. Thus, it is intended that the scope of the present inventions herein disclosed should not be limited by the particular disclosed embodiments described above, but should be determined only by a fair reading of the claims that follow.

1. A gas application device for use in surgery comprising:
a gas conduit shaped and configured as a loop to overlie a pericardial cavity of a patient;
a gas inlet port fluidly coupled to the gas conduit and configured to connect to a gas source;
a plurality of gas outlets substantially evenly spaced along the gas conduit;
a plurality of flow limiters, each of the plurality of flow limiters fluidly coupled to a corresponding one of the plurality of gas outlets;
a plurality of diffusers, each of the plurality of diffusers fluidly coupled to a corresponding one of the plurality of gas outlets;
a draping sheet mounted to an upper surface of the gas conduit and extending generally radially outward from the loop shaped gas conduit;
a plurality of suture holders formed in an upper surface of the gas conduit; and
wherein the flow limiters are configured to maintain substantially equal volumetric flow rates in each of the plurality of gas outlets, and
wherein the diffusers are configured to create microcurrents in a flow of gas exiting the gas outlets.

2. The gas application device of claim 1, wherein each of the plurality of flow limiters comprises a generally tubular body and a gas-permeable foam material disposed within the generally tubular body.

3. The gas application device of claim 1, wherein each of the plurality of flow limiters comprises:
a generally tubular body;
an obstructing element movable between an open position wherein the generally tubular body admits gas flow and a closed position wherein the generally tubular body rejects gas flow; and
a spring operatively connecting the obstructing element to the generally tubular body and configured to bias the obstructing element into the closed position; and
wherein the spring is configured such that a predetermined gas flow pressure through the flow limiter moves the obstructing element towards the open position.

4. The gas application device of claim 1, wherein the draping sheet is formed of a plurality of draping sheet segments, and wherein one of the draping sheet segments overlaps each of two adjoining draping sheet segments such that a clearance is formed under the draping sheet segment when the gas application device is applied to a surgical site.

5. The gas application device of claim 1, further comprising a movable gas applicator fluidly coupled to the gas conduit.

6. A gas application device for use in surgery comprising:
a support structure; and
a plurality of gas outlets arranged along the support structure;
wherein each of the gas outlets is configured to operate at approximately a same volume flow rate as each other of the plurality of gas outlets.

7. The gas application device of claim 6, wherein the support structure comprises a gas conduit.

8. The gas application device of claim 7, further comprising a plurality of flow limiters, wherein each of the plurality of flow limiters is disposed within the gas conduit and fluidly coupled to a corresponding one of the plurality of gas outlets, and wherein a flow limiting characteristic of each of the plurality of flow limiters is configured such that each of the gas outlets is configured to operate at approximately the same volume flow rate as each other of the plurality of gas outlets.

9. The gas application device of claim 8, wherein each of the plurality of flow limiters comprises a generally tubular body and a gas-permeable foam material disposed within the generally tubular body.

10. The gas application device of claim 8, wherein each of the plurality of flow limiters comprises:
a generally tubular body;
an obstructing element movable between an open position wherein the generally tubular body admits gas flow and a closed position wherein the generally tubular body rejects gas flow; and
a spring operatively connecting the obstructing element to the generally tubular body and configured to bias the obstructing element into the closed position; and
wherein the spring is configured such that a predetermined gas flow pressure through the flow limiter moves the obstructing element towards the open position.

11. The gas application device of claim 8, further comprising a plurality of diffusers, wherein each of the plurality of diffusers is disposed within the gas conduit and fluidly coupled to a corresponding one of the plurality of flow limiters.

12. The gas application device of claim 11, wherein each of the plurality of diffusers is configured to diffuse a gas flow into microcurrents.

13. The gas application device of claim 12, wherein each of the plurality of diffusers comprises a diffusion material selected from the group of microfiber, airstone, polyurethane foam, and fluid resistant foam.

14. The gas application device of claim 6, further comprising a draping sheet affixed to an outer edge of the support structure.

15. The gas application device of claim 14, wherein the draping sheet is formed of a plurality of draping sheet segments, and wherein one of the draping sheet segments overlaps an adjoining draping sheet segment such that a clearance is formed under the one draping sheet segment.

16. The gas application device of claim 14, wherein the draping sheet comprises a pouch formed in an upper surface of the draping sheet.

17. The gas application device of claim 6, further comprising a plurality of suture holders disposed on an upper surface of the support structure.

18. The gas application device of claim 17, wherein each of the plurality of suture holders is a groove formed in an upper surface of the support structure.

19. (canceled)

20. (canceled)

21. The gas application device of claim 17, wherein the plurality of suture holders comprises a coil spring affixed to an upper surface of the support structure.

22. The gas application device of claim 7, further comprising a movable gas applicator fluidly coupled to the gas conduit.

23. The gas application device of claim 6, further comprising a gas filter fluidly coupled to the gas outlets and disposed upstream of the gas outlets.

24. The gas application device of claim 6, further comprising a gas humidifier fluidly coupled to the gas outlets and disposed upstream of the gas outlets.

25. (canceled)

26. The gas application device of claim 6, further comprising a gas inlet fluidly coupled to the gas outlets and located upstream of the gas outlets, the gas inlet configured to be fluidly coupled to a source of carbon dioxide gas.

27. (canceled)

28. The gas application device of claim 6, wherein the support structure is sized and configured to overlie a predetermined surgical site while allowing access to that surgical site.

29. The gas application device of claim 28, wherein the support structure is shaped as a generally rectangular loop sized and configured to overlie a pericardial cavity of an opened sternum.

30. A gas application device for use in surgery comprising:
a gas conduit loop;
a plurality of gas outlets disposed towards an interior of the loop;
wherein each of the gas outlets is configured to operate at approximately a same volume flow rate as each other of the plurality of gas outlets.

31-62. (canceled)
